(19) United States
(12) Patent Application Publication
(10) Pub. No.: US 2022/0305120 A1
(43) Pub. Date: Sep. 29, 2022

(54) MUCOSAL VACCINE FORMULATIONS

(71) Applicant: GLAXOSMITHKLINE BIOLOGICALS SA, Rixensart (BE)

(72) Inventors: Simona GALLORINI, Siena (IT); Federico NAPOLITANO, Rome (IT); Alessandra VITELLI, Rome (IT)

(73) Assignee: GLAXOSMITHKLINE BIOLOGICALS SA, Rixensart (BE)

(21) Appl. No.: 17/618,283

(22) PCT Filed: Jun. 9, 2020
(86) PCT No.: PCT/IB2020/055411

§ 371 Date: Dec. 10, 2021

Related U.S. Application Data
(60) Provisional application No. 62/859,813, filed on Jun. 11, 2019.

(51) Int. Cl. A61K 39/39; A61K 47/10; A61K 47/32; A61K 47/38; A61P 31/14

(52) U.S. Cl. CPC A61K 39/39; A61K 47/10; A61K 47/32; A61K 47/38; A61P 31/14; A61K 2039/5256

(57) ABSTRACT

Simian adenoviral vectors are formulated with bioadhesives and excipients that maintain immunogenicity. They can be administered mucosally to provide effective prophylaxis and therapy.

### FIELD OF THE INVENTION

The invention is in the field of preventing and treating diseases. In particular, the invention relates to formulations suitable for the mucosal administration of simian adenoviral vaccines.

### BACKGROUND OF THE INVENTION

Adenoviral vectors have been demonstrated to provide prophylactic and therapeutic delivery platforms whereby a nucleic acid sequence encoding a therapeutic molecule is incorporated into the adenoviral genome and expressed when the adenoviral particle is administered to the treated subject. Most humans are exposed to and develop immunity to human adenoviruses. Thus there is a demand for vectors which effectively deliver prophylactic or therapeutic molecules to a human subject while minimizing the effect of pre-existing immunity to human adenovirus serotypes. Simian adenoviruses are effective in this regard because humans have little or no pre-existing immunity to the simian viruses, yet these viruses are sufficiently closely related to human viruses to be effective in eliciting potent immune responses that are minimally affected by pre-existing immunity (Vitelli et al. (2017) Expert Rev Vaccines 16:1241).

Vaccination is one of the most effective methods for preventing infectious diseases. Vaccines are typically administered via an intramuscular route, however alternative delivery routes, e.g., intradermal, oral, mucosal and others have been reported. Delivery of adenovirus-based vaccines by mucosal routes has been shown to circumvent the effect of pre-existing immunity and induce a significant immune response against an encoded antigen. For example, a human adenovirus expressing Ebola and delivered orally or intranasally protected against an Ebola challenge (Croyle et al. (2008) PLoS One 3:e3548).

However, formulating adenoviral vaccines for mucosal administration poses challenges. The adenoviruses must be administered at high concentrations to achieve an effective dose in the small volumes necessary and must remain stable at these high concentrations. The viscosity of the vaccine must be sufficient to maintain contact with the mucosa. With respect to sublingual administration, proteases in saliva degrade the vaccines; saliva can cause some of the vaccine to be swallowed, thus lost to the sublingual mucosa; and the surface area of the sublingual epithelium is relatively small. Retention is difficult and considerable effort is required to keep the vaccine in contact with the epithelium. Thus, there is a need in the art for an effective, stable vaccine formulation that can be administered mucosally.

### SUMMARY OF THE INVENTION

The invention provides vaccine formulations with bioadhesive polymers that increase the retention and consequently the absorption and penetration of a viral vaccine vector. The invention also provides the delivery of adenovirus via mucosal routes to induce antigen specific humoral and cellular immune responses.

In an embodiment, the invention provides a composition comprising a recombinant simian adenovirus encoding an immunogenic transgene and a bioadhesive excipient in an aqueous formulation comprising a simian adenovirus and one or more bioadhesives. The formulation may comprise an amorphous sugar. In specific embodiments, the amorphous sugar may be trehalose or sucrose. It may comprise a low concentration of a salt. In specific embodiments, the bioadhesive may be a poloxamer, e.g., a Pluronic, e.g., Pluronic F-68, Pluronic 127 or Poloxamer 407; a carbomer, hydroxypropylmethylcellulose; water-soluble chitosan or carboxymethylcellulose (CMC). In more specific embodiments, the bioadhesive is CMC or Poloxamer 407. In an even more specific embodiment, the concentration of CMC is 0.25% to 5.0%, 0.5% to 5.0%, e.g., 0.5% to 4.0%. 0.5% to 3.0%, 0.5% to 2.5%, 0.75% to 4.0%, 0.75% to 3.0%, 0.75% to 2.5%, 1.0% to 4.0%. 1.0% to 3.0%. 1.0% to 2.5%. 1.0%-2.0%, 1.25%-1.75% or 1.5% w/v. In another specific embodiment, the concentration of Poloxamer 407 is 10% to 30%, e.g.,10% to 25%, 15% to 30%, 15% to 25%, 15% to 20%, 20% to 25%, 18% to 22%, 19% to 21% or 20% (w/v).

In an embodiment of the invention, the vectors can be administered mucosally. In an embodiment of the invention, the vectors can be administered sublingually. In an embodiment of the invention, the vectors can be administered buccally.

In an embodiment of the invention, the adenovirus is administered in a small volume. Accordingly, the adenovirus is highly concentrated, e.g. in immunologically effective concentrations. The adenovirus can be administered at, i.e., the concentration of adenovirus in a composition of the invention is 10¹² vp/ml, 10¹¹ vp/ml, 10¹⁰ vp/ml, 10⁹ vp/ml or 10⁸ vp/ml.

In an embodiment of the invention, the adenovirus is formulated with a bioadhesive. In an embodiment the adenovirus is formulated with Tris buffer. In an embodiment, the adenovirus is formulated with histidine. In an embodiment, the adenovirus is formulated with sodium chloride. In an embodiment, the adenovirus is formulated with magnesium chloride. In an embodiment of the invention, the adenovirus is formulated with an amorphous sugar. In an embodiment, the adenovirus is formulated with a surfactant. In an embodiment, the adenovirus is formulated with vitamin E succinate (VES). In an embodiment, the adenovirus is formulated with albumin. In an embodiment, the adenovirus is formulated with ethanol. In an embodiment, the adenovirus is formulated with ethylenediaminetetraacetic acid (EDTA). In an embodiment, the adenovirus is formulated with polyethylene glycol (PEG).

In an embodiment of the invention, the simian adenovirus is formulated with one or more bioadhesives at higher viral concentrations than typically found in injectable liquid concentrations.

### DESCRIPTION OF THE DRAWINGS

FIG. 1 Stability of simian adenovirus determined by infectivity and measured by hexon-ELISA in HEK293 cells. The virus was formulated in Formulation 1 (circles), Formulation 2 (squares), Formulation 2 with 1.5% CMC (triangles) or Formulation 2 with 20% Pluronic (inverted triangles) and incubated at 4° C. for six months. The number of infectious particles per ml (ip/ml) was determined at 14, 30, 60, 90, 120, 150 and 180 days.

FIG. 2 Immunogenicity of simian adenovirus in mice after sublingual (SL) or intramuscular (IM) administration of adenovirus comprising a rabies transgene (ChAd155-RG). Virus neutralizing antibodies were measured at week 4 (circles), week 8 (squares) and 12 (triangles). The dotted line shows the seroconversion threshold of anti-rabies immunity.

FIG. 3 Systemic IgG response to simian adenovirus in mice after sublingual (SL) administration in the presence or absence of an adjuvant and after intranasal (IN) administration. Serum IgG was measured at week 4 (post-prime), week 7 (pre-boost) and week 8 (post-boost). The bars indicate the IgG serum titers to the RSV pre-F transgene.

FIG. 4 Systemic neutralizing antibody response to simian adenovirus in mice after sublingual

(SL) or intranasal (IN) administration of adenovirus comprising the RSV pre-F transgene. Virus neutralizing antibodies were measured at week 4 (open columns) and week 8 (hatched columns) and expressed as ED₆₀. The dotted line shows the limit of detection and the numbers above the bars denote the ED₆₀.

FIG. 5 Secretory IgA (sIgA) response to simian adenovirus in mice after sublingual (SL) administration or intranasal (IN) administration in the presence or absence of adjuvant. Secretory IgA was measured in saliva by ELISA at week 4 (post-prime) and week 8 (one-week post-boost). The bars indicate the optical density at 405 nm, corresponding to the sIgA titer.

FIG. 6 T cell response to simian adenovirus was measured at week four (post-prime) and week eight (post-boost) in the spleen and the lung of mice by IFNγ ELISpot after sublingual (SL) or intranasal (IN) administration. Results are expressed as spot forming units per 10⁶ lymphocytes.

FIG. 7 Systemic IgG response to simian adenovirus in mice after sublingual administration in the presence or absence of an adjuvant and after intranasal or intramuscular administration. Serum IgG was measured at week 4, week 8, week 12 (pre-boost) and week 13 (post-boost). The bars indicate the anti-pre F IgG serum titers.

FIG. 8 Systemic neutralizing antibody response to simian adenovirus in mice after sublingual (SL) or intranasal (IN) administration of the virus comprising the RSV pre-F transgene. Virus neutralizing antibodies were measured at week 4 (open bars), week 8 (light stipple), week 12 (pre-boost) (medium stipple) and week 13 (post-boost) (dark stipple). The bars indicate the anti-pre F IgG serum titers.

FIG. 9 Secretory IgA response to simian adenovirus in mice after sublingual (SL) administration in the presence or absence of adjuvant, intranasal (IN) administration or intramuscular (IM) administration. Secretory IgA was measured at week 4 and week 13 (post-boost). The bars indicate the optical density at 450 nm, corresponding to the sIgA titer.

FIG. 10 Serum (systemic) IgA levels following serum depletion of IgG. Serum IgA titer was measured at week 4, week 8, week 12 (pre-boost) and week 13 (post-boost). The bars indicate the optical density at 450 nm, corresponding to the serum IgA titers.

FIG. 11 T cell response to simian adenovirus was measured in the spleen and the lung of mice by IFNγ ELISpot after sublingual (SL) or intranasal (IN) administration. Results are expressed as spot forming units per 10⁶ lymphocytes.

### DETAILED DESCRIPTION OF THE INVENTION

### Constructs, Antigens And Variants

The present invention provides constructs useful as components of immunogenic compositions for the induction of an immune response in a subject against diseases caused by infectious pathogenic organisms. These constructs are useful for the expression of antigens, methods for their use in treatment, and processes for their manufacture. A “construct” is a genetically engineered molecule. A “nucleic acid construct” refers to a genetically engineered nucleic acid and may comprise RNA or DNA, including non-naturally occurring nucleic acids. In some embodiments, the constructs disclosed herein encode wild-type polypeptide sequences, variants or fragments thereof of pathogenic organisms, e.g., viruses, bacteria, fungi, protozoa or parasite.

A composition of the invention may be administered with or without an adjuvant. Alternatively or additionally, the composition may comprise, or be administered in conjunction with, one or more adjuvants (e.g. vaccine adjuvants).

As used herein, the term “antigen” refers to a molecule containing one or more epitopes (e.g., linear, conformational or both) that will stimulate a hosts immune system to make a humoral response, i.e., B cell mediated antibody production, and/or a cellular antigen-specific immunological response, i.e. T cell mediated immunity. An “epitope” is that portion of an antigen that determines its immunological specificity.

A “variant” of a polypeptide sequence includes amino acid sequences having one or more amino acid additions, substitutions and/or deletions when compared to the reference sequence. The variant may comprise an amino acid sequence which is at least 70%, at least 75%, at least 80%, at least 85%, at least 90%, at least 95%, at least 96%, at least 97%, at least 98%, or at least 99% identical to a full-length wild-type polypeptide. Alternatively, or in addition to, a fragment of a polypeptide may comprise an immunogenic fragment (i.e. an epitope-containing fragment) of the full-length polypeptide which may comprise or consist of a contiguous amino acid sequence of at least 6, at least 7, at least 8, at least 9, at least 10, at least 11, at least 12, at least 13, at least 14, at least 15, at least 16, at least 17, at least 18, at least 20, or more amino acids which is identical to a contiguous amino acid sequence of the full-length polypeptide.

For the purposes of comparing two closely-related polynucleotide or polypeptide sequences, the “% identity” between a first sequence and a second sequence may be calculated using an alignment program, such as BLAST® (available at blast.ncbi.nlm.nih.gov, last accessed 9 Mar. 2015) using standard settings. The % identity is the number of identical residues divided by the number of residues in the reference sequence, multiplied by 100. The % identity figures referred to above and in the claims are percentages calculated by this methodology. An alternative definition of % identity is the number of identical residues divided by the number of aligned residues, multiplied by 100. Alternative methods include using a gapped method in which gaps in the alignment, for example deletions in one sequence relative to the other sequence, are accounted for in a gap score or a gap cost in the scoring parameter. For more information, see the BLAST® fact sheet available at ftp.ncbi.nlm.nih.gov/pub/factsheets/HowTo_BLASTGuide.pdf, last accessed on 9 Mar. 2015.

Sequences that preserve the functionality of the polynucleotide or a polypeptide encoded thereby are likely to be more closely identical. Polypeptide or polynucleotide sequences are said to be the same as or identical to other polypeptide or polynucleotide sequences, if they share 100% sequence identity over their entire length.

A “difference” between sequences refers to an insertion, deletion or substitution of a single amino acid residue in a position of the second sequence, compared to the first sequence. Two polypeptide sequences can contain one, two or more such amino acid differences. Insertions, deletions or substitutions in a second sequence which is otherwise identical (100% sequence identity) to a first sequence result in reduced percent sequence identity. For example, if the identical sequences are 9 amino acid residues long, one substitution in the second sequence results in a sequence identity of 88.9%. If the identical sequences are 17 amino acid residues long, two substitutions in the second sequence results in a sequence identity of 88.2%. If the identical sequences are 7 amino acid residues long, three substitutions in the second sequence results in a sequence identity of 57.1%. If first and second polypeptide sequences are 9 amino acid residues long and share 6 identical residues, the first and second polypeptide sequences share greater than 66% identity (the first and second polypeptide sequences share 66.7% identity). If first and second polypeptide sequences are 17 amino acid residues long and share 16 identical residues, the first and second polypeptide sequences share greater than 94% identity (the first and second polypeptide sequences share 94.1% identity). If first and second polypeptide sequences are 7 amino acid residues long and share 3 identical residues, the first and second polypeptide sequences share greater than 42% identity (the first and second polypeptide sequences share 42.9% identity).

Alternatively, for the purposes of comparing a first, reference polypeptide sequence to a second, comparison polypeptide sequence, the number of additions, substitutions and/or deletions made to the first sequence to produce the second sequence may be ascertained. An addition is the addition of one amino acid residue into the sequence of the first polypeptide (including addition at either terminus of the first polypeptide). A substitution is the substitution of one amino acid residue in the sequence of the first polypeptide with one different amino acid residue. A deletion is the deletion of one amino acid residue from the sequence of the first polypeptide (including deletion at either terminus of the first polypeptide).

For the purposes of comparing a first, reference polynucleotide sequence to a second, comparison polynucleotide sequence, the number of additions, substitutions and/or deletions made to the first sequence to produce the second sequence may be ascertained. An addition is the addition of one nucleotide residue into the sequence of the first polynucleotide (including addition at either terminus of the first polynucleotide). A substitution is the substitution of one nucleotide residue in the sequence of the first polynucleotide with one different nucleotide residue. A deletion is the deletion of one nucleotide residue from the sequence of the first polynucleotide (including deletion at either terminus of the first polynucleotide).

Suitably substitutions in the sequences of the present invention may be conservative substitutions. A conservative substitution comprises the substitution of an amino acid with another amino acid having a chemical property similar to the amino acid that is substituted (see, for example, Stryer et al, Biochemistry, 5^{th} Edition 2002, pages 44-49). Preferably, the conservative substitution is a substitution selected from the group consisting of: (i) a substitution of a basic amino acid with another, different basic amino acid; (ii) a substitution of an acidic amino acid with another, different acidic amino acid; (iii) a substitution of an aromatic amino acid with another, different aromatic amino acid; (iv) a substitution of a non-polar, aliphatic amino acid with another, different non-polar, aliphatic amino acid; and (v) a substitution of a polar, uncharged amino acid with another, different polar, uncharged amino acid. A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophan. A non-polar, aliphatic amino acid is preferably selected from the group consisting of glycine, alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

Alternatively or additionally, the cross-protective breadth of a vaccine construct can be increased by comprising a medoid sequence of an antigen. By “medoid” is meant a sequence with a minimal dissimilarity to other sequences. Alternatively or additionally, a vector of the invention comprises a medoid sequence of a protein or immunogenic fragment thereof. Alternatively or additionally, the medoid sequence is derived from a natural viral strain with the highest average percent of amino acid identity among all related protein sequences annotated in the NCBI database.

As a result of the redundancy in the genetic code, a polypeptide can be encoded by a variety of different nucleic acid sequences. Coding is biased to use some synonymous codons, i.e., codons that encode the same amino acid, more than others. By “codon optimized” it is meant that modifications in the codon composition of a recombinant nucleic acid are made without altering the amino acid sequence. Codon optimization has been used to improve mRNA expression in different organisms by using organism-specific codon-usage frequencies.

In addition to, and independently from, codon bias, juxtaposition of codons in open reading frames is not random and some codon pairs are used more frequently than others. This codon pair bias means that some codon pairs are overrepresented and others are underrepresented. By “codon pair optimized,” it is meant that modifications in the codon pairing are made without altering the amino acid sequence of the individual codons. Constructs of the invention can comprise a codon optimized nucleic acid sequence and/or a codon pair optimized nucleic acid sequence

By “polypeptide” is meant a plurality of covalently linked amino acid residues defining a sequence and linked by amide bonds. The term is used interchangeably with “peptide” and “protein” and is not limited to a minimum length of the polypeptide. The term polypeptide also embraces post-translational modifications introduced by chemical or enzyme-catalyzed reactions, as are known in the art. The term can refer to fragments of a polypeptide or variants of a polypeptide such as additions, deletions or substitutions.

A polypeptide of the invention can be in a non-naturally occurring form (e.g. a recombinant or modified form). Polypeptides of the invention can have covalent modifications at the C-terminus and/or N-terminus. They can also take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.). The polypeptides can be naturally or non-naturally glycosylated (i.e. the polypeptide may have a glycosylation pattern that differs from the glycosylation pattern found in the corresponding naturally occurring polypeptide).

The skilled person will recognise that individual substitutions, deletions or additions to a protein which alters, adds or deletes a single amino acid or a small percentage of amino acids is an “immunogenic derivative” where the alteration(s) results in the substitution of an amino acid with a functionally similar amino acid or the substitution/deletion/addition of residues which do not impact the immunogenic function.

Conservative substitution tables providing functionally similar amino acids are well known in the art. In general, such conservative substitutions will fall within one of the amino-acid groupings specified below, though in some circumstances other substitutions may be possible without substantially affecting the immunogenic properties of the antigen. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan
7) Serine (S), Threonine (T); and
8) Cysteine (C), Methionine (M)

Suitably such substitutions do not occur in the region of an epitope, and do not therefore have a significant impact on the immunogenic properties of the antigen.

Immunogenic derivatives may also include those wherein additional amino acids are inserted compared to the reference sequence. Suitably such insertions do not occur in the region of an epitope, and do not therefore have a significant impact on the immunogenic properties of the antigen. One example of insertions includes a short stretch of histidine residues (e.g. 2-6 residues) to aid expression and/or purification of the antigen in question.

Immunogenic derivatives include those wherein amino acids have been deleted compared to the reference sequence. Suitably such deletions do not occur in the region of an epitope, and do not therefore have a significant impact on the immunogenic properties of the antigen. The skilled person will recognise that a particular immunogenic derivative may comprise substitutions, deletions, insertions and additions (or any combination thereof).

### Adenoviruses

Adenoviruses are nonenveloped icosahedral viruses with a linear double stranded DNA genome of approximately 36 kb. Adenoviruses can transduce numerous cell types of several mammalian species, including both dividing and nondividing cells, without integrating into the genome of the host cell. Human adenoviral vectors are currently used in gene therapy and vaccines but have the drawback of a high worldwide prevalence of pre-existing immunity, following previous exposure to common human adenoviruses. Simian adenoviruses have the advantage that they are sufficiently closely related to human viruses that they can enter into human cells and deliver transgenes, but humans have little or no pre-existing immunity.

Adenoviruses have a characteristic morphology with an icosahedral capsid comprising three major proteins, hexon (II), penton base (III) and a knobbed fiber (IV), along with a number of other minor proteins, VI, VIII, IX, IIIa and IVa2. The hexon accounts for the majority of the structural components of the capsid, which consists of 240 trimeric hexon capsomeres and 12 penton bases. The hexon has three conserved double barrels and the top has three towers, each tower containing a loop from each subunit that forms most of the capsid. The base of the hexon is highly conserved between adenoviral serotypes, while the surface loops are variable. The penton is another adenoviral capsid protein; it forms a pentameric base to which the fiber attaches. The trimeric fiber protein protrudes from the penton base at each of the 12 vertices of the capsid and is a knobbed rod-like structure. The primary role of the fiber protein is to tether the viral capsid to the cell surface via the interaction of the knob region with a cellular receptor. Variations in the flexible shaft, as well as knob regions of fiber, are characteristic of the different adenoviral serotypes.

The adenoviral genome has been well characterized. The linear, double-stranded DNA is associated with the highly basic protein VII and a small peptide pX (also termed mu). Another protein, V, is packaged with this DNA-protein complex and provides a structural link to the capsid via protein VI. There is general conservation in the overall organization of the adenoviral genome with respect to specific open reading frames being similarly positioned, e.g. the location of the E1A, E1B, E2A, E2B, E3, E4, L1, L2, L3, L4 and L5 genes of each virus. Each extremity of the adenoviral genome comprises a sequence known as an inverted terminal repeat (ITR), which is necessary for viral replication. The 5′ end of the adenoviral genome contains the 5′ cis-elements necessary for packaging and replication; i.e., the 5′ ITR sequences (which can function as origins of replication) and the native 5′ packaging enhancer domains, which contain sequences necessary for packaging linear adenoviral genomes and enhancer elements for the E1 promoter. The 3′ end of the adenoviral genome includes 3′ cis-elements, including the ITRs, necessary for packaging and encapsidation. The virus also comprises a virus-encoded protease, which is necessary for processing some of the structural proteins required to produce infectious virions.

The structure of the adenoviral genome is described on the basis of the order in which the viral genes are expressed following host cell transduction. The viral genes are referred to as early (E) or late (L) genes according to whether transcription occurs prior to or after onset of DNA replication. In the early phase of transduction, the E1A, E1B, E2A, E2B, E3 and E4 genes of adenovirus are expressed to prepare the host cell for viral replication. The E1 gene is considered a master switch, it acts as a transcription activator and is involved in both early and late gene transcription. E2 is involved in DNA replication; E3 is involved in immune modulation and E4 regulates viral mRNA metabolism. During the late phase of infection, expression of the late genes L1-L5, which encode the structural components of the viral particles, is activated. Late genes are transcribed from the Major Late Promoter (MLP) with alternative splicing.

Historically, adenovirus vaccine development has focused on defective, non-replicating vectors. They are rendered replication defective by deletion of the E1 region genes, which are essential for replication. Typically, non-essential E3 region genes are also deleted to make room for exogenous transgenes. E4 region genes may also be deleted. An expression cassette comprising the transgene under the control of an exogenous promoter is then inserted. These replication-defective viruses are then produced in E1-complementing cells. Replication competent adenoviruses have also been described (WO 2019/076877). Adenoviruses of the invention include both replication competent and replication defective simian adenoviruses.

The term “replication-defective ” or “replication-incompetent” adenovirus refers to an adenovirus that is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc., or a complete removal of a gene encoding a gene product that is essential for viral replication, such as one or more of the adenoviral genes selected from E1A, E1B, E2A, E2B, E3 and E4 (such as E3 ORF1, E3 ORF2, E3 ORF3, E3 ORF4, E3 ORF5, E3 ORF6, E3 ORF7, E3 ORF8, E3 ORF9, E4 ORF7, E4 ORF6, E4 ORF4, E4 ORF3, E4 ORF2 and/or E4 ORF1). Suitably, E1 and optionally E3 and/or E4 are deleted. If deleted, the aforementioned deleted gene region will suitably not be considered in the alignment when determining percent identity with respect to another sequence.

The term “replication-competent” adenovirus refers to an adenovirus which can replicate in a host cell in the absence of any recombinant helper proteins comprised in the cell. Suitably, a “replication-competent” adenovirus comprises intact structural genes and the following intact or functionally essential early genes: E1A, E1B, E2A, E2B and E4. Wild type adenoviruses isolated from a particular animal will be replication competent in that animal.

### Vectors of the Invention

A “vector” refers to a nucleic acid that has been substantially altered relative to a wild type sequence and/or incorporates a heterologous sequence, i.e., nucleic acid obtained from a different source, and replicating and/or expressing the inserted polynucleotide sequence, when introduced into a cell (i.e., a “host cell”). In the case of replication defective adenoviruses, the host cell may be E1, E3 or E4 complementing. A vector of the invention may include any genetic element, including naked DNA, a phage, transposon, cosmid, episome, plasmid or viral component. In embodiments of the adenoviral vectors of the invention, the adenoviral DNA is capable of entering a mammalian target cell, i.e. it is infectious.

Vectors of the invention may contain simian adenoviral DNA. In one embodiment, the adenoviral vector of the invention is derived from a nonhuman simian adenovirus, also referred to as a “simian adenovirus.” Numerous adenoviruses have been isolated from nonhuman simians such as chimpanzees, bonobos, rhesus macaques, orangutans and gorillas. Vectors derived from these adenoviruses can induce strong immune responses to transgenes encoded by these vectors. Certain advantages of vectors based on nonhuman simian adenoviruses include a relative lack of cross-neutralizing antibodies to these adenoviruses in the human target population, thus their use overcomes the pre-existing immunity to human adenoviruses.

Adenoviral vectors of the invention may be derived from a non-human simian adenovirus, e.g., from chimpanzees (Pan troglodytes), bonobos (Pan paniscus), gorillas (Gorilla gorilla) and orangutans (Pongo abelii and Pongo pygnaeus). They include adenoviruses from Group B, Group C, Group D, Group E and Group G. Chimpanzee adenoviruses include, but are not limited to ChAd3, ChAd15, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd157, ChAdOx1, ChAdOx2 and SAdV41. Alternatively, adenoviral vectors may be derived from nonhuman simian adenoviruses isolated from bonobos, such as PanAd1, PanAd2, PanAd3, Pan 5, Pan 6, Pan 7 (also referred to as C7) and Pan 9 or gorillas such as GADNOU19 and GADNOU20. Vectors may include, in whole or in part, a nucleotide encoding the fiber, penton or hexon of a non-human adenovirus.

In an embodiment of the invention, the vector is a functional or an immunogenic derivative of an adenoviral vector. By “derivative of an adenoviral vector” is meant a modified version of the vector, e.g., one or more nucleotides of the vector are deleted, inserted, modified or substituted. Such simian adenoviral vectors are derived from molecular clones in which the viral genome is carried by a plasmid vector. The use of vectors derived from bacterial plasmids eliminates the risk of possible contamination with unidentified pathogens that could propagate unnoticed in cell culture and cause harm to a human recipient.

As set forth above, the choice of gene expression cassette insertion sites of replication defective vectors has been primarily focused on replacing regions known to be involved in viral replication. The choice of gene expression cassette insertion sites of replication competent vectors must preserve the replication machinery. Consequently, replication competent viral vectors must preserve the sequences necessary for replication while allowing room for functional expression cassettes.

Regulatory elements of a vector of the invention, i.e., expression control sequences, include appropriate transcription initiation, termination, promoter and enhancer sequences; efficient RNA processing signals such as splicing and polyadenylation (poly A) signals including rabbit beta-globin polyA; tetracycline regulatable systems, microRNAs, posttranscriptional regulatory elements e.g., WPRE, posttranscriptional regulatory element of woodchuck hepatitis virus); sequences that stabilize cytoplasmic mRNA; sequences that enhance translation efficiency (e.g., Kozak consensus sequence); sequences that enhance protein stability; and when desired, sequences that enhance secretion of an encoded product.

A “promoter” is a nucleotide sequence that permits the binding of RNA polymerase and directs the transcription of a gene. Typically, a promoter is located in a non-coding region of a gene, proximal to the transcriptional start site. Sequence elements within promoters that function in the initiation of transcription are often characterized by consensus nucleotide sequences. Examples of promoters include, but are not limited to, promoters from bacteria, yeast, plants, viruses, and mammals, including simians and humans. A great number of expression control sequences, including promoters which are internal, native, constitutive, inducible and/or tissue-specific, are known in the art and may be utilized.

Promoters of the invention include, but are not limited to, CMV promoters, beta-actin promoters, e.g., chicken beta actin (CAG) promoters, CASI promoters, human phosphoglycerate kinase-1(PGK) promoters, TBG promoters, retroviral Rous sarcoma virus LTR promoters, SV40 promoters, dihydrofolate reductase promoters, phosphoglycerol kinase (PGK) promoters, EF1a promoters, zinc-inducible sheep metallothionine (MT) promoters, dexamethasone (Dex)-inducible mouse mammary tumor virus (MMTV) promoters, T7 polymerase promoter systems, ecdysone insect promoters, tetracycline-repressible systems, tetracycline-inducible systems, RU486-inducible systems and rapamycin-inducible systems.

Suitable promoters include the cytomegalovirus (CMV) promoter and the CASI promoter. The CMV promoter is strong and ubiquitously active. It has the ability to drive high levels of transgene expression in many tissue types and is well known in the art. The CMV promoter can be used in vectors of the invention, either with or without a CMV enhancer. The CASI promoter is a synthetic promoter described as a combination of the CMV enhancer, the chicken beta-actin promoter, and a splice donor and splice acceptor flanking the ubiquitin (UBC) enhancer (U.S. Pat. No. 8,865,881).

A “posttranscriptional regulatory element,” as used herein, is a DNA sequence that, when transcribed, enhances the expression of the transgene(s) or fragments thereof that are delivered by viral vectors of the invention. Postranscriptional regulatory elements include, but are not limited to, the Hepatitis B Virus Postranscriptional Regulatory Element (HPRE) and the Woodchuck Hepatitis Postranscriptional Regulatory Element (WPRE). The WPRE is a tripartite cis-acting element that has been demonstrated to enhance transgene expression driven by certain, but not all, promoters.

Vectors of the invention may comprise a transgene used to deliver desired RNA or protein sequences, for example heterologous sequences, for in vivo expression. A “transgene” is a nucleic acid sequence, heterologous to the vector sequences flanking the transgene, which encodes a polypeptide of interest. The nucleic acid coding sequence is operatively linked to regulatory components in a manner which permits transgene transcription, translation, and/or expression in a host cell. In embodiments of the invention, the vectors express transgenes at a therapeutic or a prophylactic level. A “functional derivative” of a transgenic polypeptide is a modified version of a polypeptide, e.g., wherein one or more amino acids are deleted, inserted, modified or substituted. An “expression cassette” comprises a transgene and regulatory elements necessary for the translation, transcription and/or expression of the transgene in a host cell.

Optionally, vectors carrying transgenes encoding therapeutically useful or immunogenic products may also include selectable markers or reporter genes. The reporter gene may be chosen from those known in the art. Suitable reporter genes include, but are not limited, to enhanced green fluorescent protein, red fluorescent protein, luciferase and secreted embryonic alkaline phosphatase (seAP), which may include sequences encoding geneticin, hygromicin or purimycin resistance, among others. Such selectable reporters or marker genes (which may or may not be located outside the viral genome to be packaged into a viral particle) can be used to signal the presence of the plasmids in bacterial cells, such as ampicillin resistance. Other components of the vector may include an origin of replication.

In addition to the transgene, the expression cassette also includes conventional control elements which are operably linked to the transgene in a manner that permits its transcription, translation and/or expression in a cell transfected with the adenoviral vector. As used herein, “operably linked” sequences include both expression control sequences that are contiguous with the gene of interest and expression control sequences that act in trans or at a distance to control the gene of interest.

The transgene may be used for prophylaxis or treatment, e.g., as a vaccine for inducing an immune response, to correct genetic deficiencies by correcting or replacing a defective or missing gene, or as a cancer therapeutic. As used herein, induction of an immune response refers to the ability of a protein to induce a T cell and/or a humoral antibody immune response to the protein.

The immune response elicited by the transgene may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing cytokines, e.g. interferon gamma (IFN gamma), tumor necrosis factor alpha (TNF alpha) and/or interleukin 2 (IL2). Alternatively, or additionally, the antigen specific T cell response comprises a CD8+ T cell response, such as a response involving CD8+ T cells expressing cytokines, e.g., IFN gamma, TNF alpha and/or IL2.

The composition of the transgene sequence will depend upon the use to which the resulting vector will be put. In an embodiment, the transgene is a sequence encoding a product which is useful in biology and/or medicine, such as a prophylactic transgene, a therapeutic transgene or an immunogenic transgene, e.g., protein or RNA. Protein transgenes include antigens. Antigenic transgenes of the invention induce an immunogenic response to a disease-causing organism. RNA transgenes include tRNA, dsRNA, ribosomal RNA, catalytic RNAs, and antisense RNAs. An example of a useful RNA sequence is a sequence which extinguishes expression of a targeted nucleic acid sequence in the treated animal. A transgene sequence may include a reporter sequence, which upon expression produces a detectable signal.

Vectors of the invention are generated using techniques provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

### Modified Vaccinia Virus Ankara

Modified Vaccinia Virus Ankara (MVA) is a member of the Orthopox family derived from the dermal vaccinia strain Ankara and attenuated for use in humans. Attenuation was performed by serial passaging and as a result, there are a number of different strains or isolates, depending on the passage number. An MVA if the invention is any attenuated strain suitable for use in humans.

The genomic organization of MVA has been described (Virology (1998) 244:365). The virus is known to be highly immunogenic. It is preferred as a boosting, rather than a priming, virus and has been described as an effective booster for DNA vaccines (U.S. Pat. No. 7,384,644).

Bioadhesive Formulations Bioadhesives increase the adherence of a formulation to a biological tissue, e.g., the mucosa, and may also enhance the permeation of the formulation into the tissue. This increases the adenovirus' residence time at the mucosa. Compositions of the invention include a bioadhesive and can include one or more of a salt, an amorphous sugar, a surfactant, a bivalent metal ion, a metal ion chelator, histidine, Vitamin E Succinate (VES) and recombinant human serum albumin (rHSA) in a buffered aqueous solution.

“Bioadhesion” is the process whereby synthetic and natural macromolecules adhere to biological surfaces and “mucoadhesion” is bioadhesion when the biological surfaces are mucosal tissues. Bioadhesives of the invention allow incorporation of adenovirus into the body and offer little or no hindrance to its release from the mucosa into the systemic circulation. If bioadhesives are incorporated into pharmaceutical formulations, the absorption by mucosal cells or the release at the site may be enhanced for an extended period of time. In the case of synthetic polymers, bioadhesion and mucoadhesion can result from a number of different physicochemical interactions. Bioadhesives of the invention and their degradation products should be non-absorbable, non-irritating to mucous membranes and adhere quickly to most tissues. In some embodiments, they have some degree of site-specificity.

Bioadhesive e.g., mucoadhesive agents can improve the bioavailability of an active agent by improving the residence time at a mucosal delivery site. Preferable properties of these agents are that they are non-toxic, predominately non-absorbable, non-irritating to the mucous membrane and form strong non-covalent bonds with the epithelial cell surfaces. Preferably, they adhere quickly to the tissue, possess some specificity to the mucosa, e.g., the mucosa of the oral cavity, do not hinder release of an active vaccine component from its formulation and are stable for the shelf life of the vaccine.

Bioadhesives suitable for use in the invention include polyoxyethylene, poly(ethylene glycol)

(PEG); poly(vinyl pyrrolidone) (PVP); poly(hydroxyethyl methacrylate) (PHEMA); polymeric blends, e.g., Pluronics such as Pluronic F-68, Pluronic 127 and Poloxamer 407 (P407) (LUTROL); polyacrylates; carbomers, e.g., carbomer 910, carbomer 934, carbomer 934P, carbomer 940, carbomer 941, carbomer 971P and carbomer 974P; polycarbophil; hyaluronic acid; chitosans, e.g., chitosan, N-trimethyl chitosan (TMC) and mono-N-carboxymethyl chitosan (MCC); alginates;

guar gum; carrageenan; and polymers derived from cellulose. Cellulosics are low cost, reproducibly manufactured, and biocompatible. Cellulosic bioadhesives include carboxymethylcellulose (CMC), microcrystalline cellulose, oxidized regenerated cellulose, hydroxyethyl cellulose (HEC), hydroxypropyl cellulose (HPC), methylcellulose and sodium carboxymethylcellulose. The bioadhesive carboxymethyl cellulose (CMC) is a chemically obtained derivative of the natural cellulose polymer. It is not digestible, not toxic, and not allergenic.

Poloxamers, also known as pluronics, are nonionic triblock copolymers composed of a central hydrophobic chain of polyoxypropylene flanked by two hydrophilic chains of polyoxyethylene. Poloxamer solutions self-assemble in a temperature dependent manner and exhibit thermo-gelling behavior. Concentrated aqueous solutions of poloxamers are liquid at low temperature and form a gel at higher temperature in a reversible process. The transitions that occur in these systems depend on the polymer composition (molecular weight and hydrophilic/hydrophobic molar ratio). When mixed with water, concentrated solutions of poloxamers can form hydrogels that can be extruded easily and can act as a carrier for other particles, e.g., vectors.

Carbomers are synthetic high-molecular weight polymers of acrylic acid. They may be homopolymers of acrylic acid or crosslinked with an allyl ether of pentaerythritol, allyl ether of sucrose or allyl ether of propylene.

Chitosan is a non-toxic, biocompatible cationic biopolymer usually obtained by alkaline deacetylation from chitin. It can act as both as a mucoadhesive and to enhance permeability across the epithelia, enhancing absorption. Chitosan opens the tight junctions of the mucosal barrier and facilitates the paracellular transport of hydrophilic macromolecules. It also has chelating capacity towards metal ions and antimicrobial effects against a broad range of gram-positive and gram-negative bacteria as well as fungi. Microcrystalline chitosan (MCCh) has greater crystallinity, hydrogen bond energy and water retention than non-crystalline chitosan.

Salts suitable for use in the invention are ionic compounds that result from the neutralization reaction of an acid and a base and are composed of a related number of cations and anions such that the product is without net charge. The component ions can either be inorganic or organic, and, can be monoatomic or polyatomic. In an embodiment, the salt is NaCl. In an embodiment, the concentration of salt in the formulation is less than 100 mM, less than 75 mM, less than 50 mM, less than 25 mM, less than 10 mM, less than 7.5 mM or less than 5 mM. In a particular embodiment, the salt is NaCl at a concentration of about 5.0 mM. In another particular embodiment, the salt is NaCl at a concentration of about 75 mM.

Amorphous sugars suitable for use in the invention may be selected from sucrose, trehalose, mannose, mannitol, raffinose, lactitol, lactobionic acid, glucose, maltulose, iso-maltulose, lactulose, maltose, lactose, isomaltose, maltitol, palatinit, stachyose, melezitose, dextran, or a combination thereof. In an embodiment, the amorphous sugar is sucrose in a concentration of 5-25%, 10-20%, 25-17% or about 16%. In an embodiment, the amorphous sugar is trehalose in a concentration of 5-25%, 10-20%, 25-17% or about 16%.

Surfactants suitable for use in the invention include a surfactant selected from poloxamer surfactants (e.g. poloxamer 188), polysorbate surfactants (e.g. polysorbate 80 and/or polysorbate 20), octoxinal surfactants, polidocanol surfactants, polyoxyl stearate surfactants, polyoxyl castor oil surfactants, N-octyl-glucoside surfactants, macrogol 15 hydroxy stearate, and combinations thereof. The surfactant can be present in an amount of at least 0.001%, at least 0.005%, at least 0.01% (w/v), and/or up to 0.5% (w/v) as calculated with respect to the aqueous mixture. In an embodiment, the surfactant is selected from poloxamer surfactants (e.g. poloxamer 188) and polysorbate surfactants (e.g. polysorbate 80 and/or polysorbate 20). In an embodiment, the surfactant is polysorbate 80 in a concentration of 0.005-0.05%, 0.01-0.04%, about 0.02% or about 0.25%.

Bivalent metal ions suitable for use in the invention include Mg²⁺, Ca²⁺ or Mn²⁺. In an embodiment, the bivalent metal ion is Mg²⁺, Ca²⁺ or Mn²⁺ in the form of a salt, such as MgCl₂, MgSO₄, CaCl₂ or MnSO₄. In a particular embodiment, the bivalent metal ion is Mg²⁺. The bivalent metal ion can be present in the aqueous mixture at a concentration of between 0.05 and 5.0 mM. In an embodiment, the bivalent metal ion is the Mg²⁺ salt MgCl₂ and is present in a concentration of about 1.0 mM.

Metal ion chelators suitable for use in the invention include ethylenediamine, ethylenediaminetetraacetic acid (EDTA), histidine, glutamic acid, aspartic acid, Vitamin B12 and dimercaptosuccinic acid. In an embodiment, the metal ion chelator is present in an amount less than 0.5% (w/v), less than 0.25% (w/v), less than 0.1% (w/v) or less than 0.05% (w/v). In an embodiment, the metal ion chelator is EDTA in a concentration of 0.01-1.0 mM, 0.05-0.5 mM or about 0.1 mM.

Formulations of the invention may also optionally include histidine at a concentration of 1.0-50 mM, 5.0-25 mM or about 10 mM. Formulations of the invention may optionally include Vitamin E Succinate (VES) at a concentration of 0.005 mM-0.5 mM, 0.01-0.1 or about 0.05 mM.

Formulations of the invention may optionally include recombinant human serum albumin (rHSA) at a concentration of 0.01-1.0 mM, 0.05-0.5 mM or about 0.1 mM.

Buffers suitable for use in the invention include Tris, succinate, borate, maleate, lysine, histidine, glycine, glycylglycine, citrate, carbonate or combinations thereof. The buffer can be present in the aqueous mixture in an amount of at least 0.5 mM. The buffer can be present in the aqueous mixture in an amount of less than 50 mM. The pH of the aqueous mixture is at least 6.0 and less than 10. In an embodiment, the buffer is Tris at a pH of 6.5-9.5 or 7.0-9.0. In an embodiment the buffer is Tris pH 7.4. In an embodiment, the buffer Is Tris pH 8.4. In an embodiment, the buffer is Tris pH 8.5.

### Mucosal Immunization

“Mucosa” is the thin skin that covers the inside surface of parts of the body and produces mucus to protect them. It typically consists of one or more layers of epithelial cells overlying a layer of loose connective tissue. Mucosal tissues include buccal, colorectal, under-eyelid, gastrointestinal, lung, nasal, ocular, sublingual and vaginal tissues.

While parenteral vaccination can prevent or treat disease by inducing a systemic response, mucosal immunization induces immunity at the site of pathogen entry. Mucosal immune responses include secretory IgA and cytotoxic T cells, both of which play a crucial role. Induction of mucosal immunity typically requires effective antigen delivery to immune-inductive sites that stimulate innate immunity which, in turn, generates an adaptive immune response.

Mucosal vaccine delivery offers several advantages to intramuscular delivery of vaccines. As the mucosa is contiguous with the outside of the body, mucosal vaccines can be effective and safe at a slightly lower degree of purity compared to parenteral vaccines, thus they are easier to produce.

They are also typically effective at low doses, thus are cost-effective. Mucosal vaccines are needle-free, eliminating the pain and fear of parenteral administration, the risk of infection from re-used needles and needle-stick injuries. They do not need to be given by highly trained professionals, thus can be more easily disseminated and even self-administered.

Mucosal vaccines can be delivered into the oral cavity, e.g., sublingually, buccally or gingivally.

The sublingual and buccal mucosa have a non-keratinized epithelium while the gum mucosa is covered with keratinized epithelium similar to that of skin. Lymphoid tissues, e.g., the tonsils and adenoids, in the naso-oro-pharyngeal cavities mediate the immune response to antigens presented via these routes. These lymphoid tissues, especially the lingual tonsil can sample vaccine antigens delivered to the oral cavity mucosa to induce an immune response. The oral cavity epithelium is also rich in dendritic antigen presenting cells.

The non-keratinized epithelium of buccal and sublingual mucosa has small amounts of neutral and polar lipids such as cholesterol sulfate and glucosyl ceramides; small amounts of non-polar lipids like ceramides and acylceramides are absent. Therefore, it has greater permeability than keratinized epithelium. Vaccine delivery via the buccal route provides an antigen with access through a layer of stratified, squamous non-keratinized epithelium which is somewhat thicker than the sublingual layer. Buccal delivery also targets Langerhans cells and induces a systemic response. The sublingual mucosa, with a thickness 100-200 μm, is relatively thinner and more vascularized than the buccal mucosa (thickness 500-800 μm) and has been demonstrated to be more permeable. Antigens delivered sublingually or buccally are targeted to the Langerhans cells within the mucosa and myeloid dendritic cells in the lamina propria.

By “buccal” is meant the cheek lining. By “gingival” is meant the gums, mouth mucosa or the inner surfaces of the lips. By “sublingual” is meant the ventral surface of the tongue or the floor of the mouth below the tongue.

Vaccine delivery via the sublingual route provides an antigen with fast access through a very thin layer of stratified, squamous non-keratinized epithelium, where it targets Langerhans cells and induces a systemic response. Antigen delivered under the tongue becomes available to a dense network of dendritic cells in the sublingual mucosa. Replication competent viruses delivered sublingually bypass the liver, thus avoiding first-pass metabolism, increasing their persistence, thus potentially generating a stronger immune response.

Sublingual administration requires low volumes, reduces exposure to digestive enzymes compared to oral administration, and avoids the intestinal tract. Sublingual vaccinations have a lower risk of central nervous system complications compared with intranasal vaccines. Sublingual dosing avoids the barriers of low stomach pH and intestinal enzyme degradation as well as avoiding first-pass hepatic metabolism encountered by oral dosing. Sublingual administration can be administered in the form of drops under the tongue, with easy control of the dose and without the need for water.

Despite these advantages, to date no sublingual vaccine for an infectious disease has been licensed for human use. Variable responses have been observed with sublingual administration to date. Variables included, but were not limited to, the time of contact of the vaccine to the sublingual mucosa, viscosity and kinetics of immunogenicity. Sublingual vaccines have been shown to be safe but not always efficacious. In some cases, systemic and mucosal immune responses have been observed in response to sublingual administration (Czerkinsky et al. (2011) Human Vaccines 7:110). For example, human adenovirus in an amorphous solid formulation was immunogenic when administered sublingually to rodents (U.S. Pat. No. 9,675,550), adjuvanted ovalbumin administered sublingually induced antibody and T cell responses in mice (Cuburu et al. (2007) Vaccine 25:8598) and sublingual administration of adjuvanted influenza vaccine elicited mucosal and systemic immune responses, the latter of which were equivalent to unadjuvanted intramuscular vaccination (Gallorini et al. (2014) Vaccine 32:2382). However, sublingual immunization with attenuated vaccinia virus encoding HIV proteins was not effective in protecting against a viral challenge (Thippeshappa et al. (2016) Clin Vaccine Immunol 23:204). This body of literature also demonstrates that the formulation of the viral vaccine vector affects its stability and potency.

### Pharmaceutical Compositions, Immunogenic Compositions And Adjuvants

Compositions of the invention may be formulated into pharmaceutical compositions prior to administration to a subject. The invention provides a pharmaceutical composition comprising a composition of the invention and one or more pharmaceutically acceptable excipients.

Pharmaceutical compositions may have an osmolality of between 200 mOsm/kg and 400 mOsm/kg, e.g. between 240-360 mOsm/kg, or between 290-310 mOsm/kg. Pharmaceutical compositions may include one or more preservatives, such as thiomersal or 2-phenoxyethanol. Mercury-free compositions are preferred, and preservative-free vaccines can be prepared. Pharmaceutical compositions may be aseptic or sterile. Pharmaceutical compositions may be non-pyrogenic e.g. containing <1 EU (endotoxin unit) per dose, and preferably <0.1 EU per dose. Pharmaceutical compositions may be gluten free. Pharmaceutical compositions may be prepared in unit dose form. Alternatively or additionally, a unit dose may have a volume of between 0.1 -2.0 ml, e.g. about 1.0 or 0.5 ml.

Compositions of the invention can be delivered via any known dosage form. These include, but are not limited to tablets, ointments, gels, patches and films.

A composition of the invention may be administered with or without an adjuvant. Alternatively or additionally, the composition may comprise, or be administered in conjunction with, one or more adjuvants (e.g. vaccine adjuvants).

By “adjuvant” is meant an agent that augments, stimulates, activates, potentiates or modulates the immune response to an active ingredient of the composition. The adjuvant effect may occur at the cellular or humoral level or both. Adjuvants stimulate the response of the immune system to the actual antigen but have no immunological effect themselves. Alternatively or additionally, adjuvented compositions of the invention may comprise one or more immunostimulants. By “immunostimulant” it is meant an agent that induces a general, temporary increase in a subject's immune response, whether administered with the antigen or separately.

Adjuvants of the invention may increase the mucosal and/or the systemic immune response.

They can include, e.g., the E. coli heat-labile enterotoxin mutant LTK63, alpha-galactosylceramide (α-GalCer) and monophosphoryl lipid A (MPL). LTK63 is a non-toxic mutant of the heat labile enterotoxin LT. The mutation eliminates the LT ADP-ribosylating activity and associated toxicity, while retaining adjuvant activity. LTK63 is known as a potent mucosal adjuvant for nasal delivery of protein antigens, enhancing antigen-specific serum immunoglobulin G (IgG), secretory IgA, and local and systemic T-cell responses. It also promotes a Th17 response to vaccine antigens after mucosal immunization; this action has a critical role in protecting against a variety of pathogens at mucosal surface. α-GalCer is a potent and specific activator of natural killer (NK) T cells and an effective adjuvant for mucosal administration of viral vectored vaccines and for protection against mucosally transmitted pathogens. Within hours of administration of α-GalCer, NK cells produce copious amounts of both regulatory and proinflammatory cytokines. MPL is a Toll-like receptor agonist.

### Methods Of Use/Uses

Methods are provided for inducing an immune response against a pathogenic organism in a subject in need thereof comprising a step of administering an immunologically effective amount of a construct or composition as disclosed herein. Some embodiments provide the use of the constructs or compositions disclosed herein for inducing an immune response to an antigen in a subject in need thereof. Some embodiments provide the use of the construct or composition as disclosed herein in the manufacture of a medicament inducing an immune response to an antigen in a subject.

In one aspect, the invention provides a composition of the invention for use as a therapeutic, prophylactic or ameliorator of a disease or disorder. In another aspect, the invention provides a composition of the invention for use in the treatment, prophylaxis or amelioration of a disease or disorder. In a further aspect, the invention provides a composition of the invention for the manufacture of a medicament for the treatment, prophylaxis or amelioration of a disease or disorder. In yet a further aspect, the invention provides a method of treatment of a disease or disorder which comprises administering to a subject in need thereof an effective amount of a composition of the invention.

Methods of the invention induce a protective immune response by immunizing or vaccinating a subject. The invention may therefore be applied for the prophylaxis, treatment or amelioration of diseases caused by an infectious agent.

A composition of the invention may be employed alone or in combination with other therapeutic agents. Combination therapies according to the invention comprise the administration of at least one composition of the invention and the use of at least one other therapeutically active agent. A composition of the invention and the other therapeutic agent(s) may be administered together in a single pharmaceutical composition or separately. When administered separately, this may occur simultaneously or sequentially in any order.

By “subject” is meant a mammal, e.g. a human or a veterinary mammal. In some embodiments the subject is human.

By “priming” is meant the administration of an immunogenic composition which induces a higher level of an immune response, when followed by a subsequent administration of the same or of a different immunogenic composition, than the immune response obtained by administration with a single immunogenic composition.

By “boosting” is meant the administration of a subsequent immunogenic composition after the administration of a priming immunogenic composition, wherein the subsequent administration produces a higher level of immune response than an immune response to a single administration of an immunogenic composition.

By “heterologous prime boost” is meant priming the immune response with an antigen and subsequent boosting of the immune response with an antigen delivered by a different molecule and/or vector. For example, heterologous prime boost regimens of the invention include priming with an RNA molecule and boosting with an adenoviral vector as well as priming with an adenoviral vector and boosting with an RNA molecule.

Am “immunologically effective amount” is the amount of an active component sufficient to elicit either an antibody or a T cell response or both sufficient to have a beneficial effect on the subject.

### Kits

The invention provides a pharmaceutical kit for the ready administration of an immunogenic, prophylactic or therapeutic regimen for treating a disease or condition caused by a pathogenic organism. The kit may be designed for use in a method of inducing an immune response by administering a priming vaccine comprising an immunologically effective amount of one or more antigens encoded by a simian adenoviral vector and subsequently administering a boosting vaccine comprising an immunologically effective amount of one or more simian adenovirus encoded antigens.

The kit contains at least one immunogenic composition comprising a simian adenoviral vector encoding an antigen. The kit may contain multiple prepackaged doses of each of the component vectors for multiple administrations of each. Components of the kit may be contained in vials. The kit also contains instructions for using the immunogenic compositions in the prime/boost methods described herein. It may also contain instructions for performing assays relevant to the immunogenicity of the components. The kit may also contain excipients, diluents, adjuvants, syringes, other appropriate means of administering the immunogenic compositions or decontamination or other disposal instructions.

Vectors of the invention are generated using techniques and sequences provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” in relation to a numerical value is optional and means, e.g., the amount ±10%.

The term “comprising” encompasses “including” as well as “consisting,” e.g., a composition comprising X may consist exclusively of X or may include something additional, e.g., X+Y. The term “substantially” does not exclude “completely.” For example, a composition that is substantially free from Z may be completely free from Z.

The present invention will now be further described by means of the following non-limiting examples.

### EXAMPLES

### Example 1: In Vitro Stability of Adenovirus in Bioadhesive Formulations

Bioadhesive formulations of adenovirus were tested in vitro for stability at 4° C. and 37° C. and after freeze-thaw. Stability was measured by qualitative PCR (qPCR) and with an infectivity assay that detects adenoviral hexon protein in cultured cells. The effect of the bioadhesive reagents on adenoviral stability was assessed using a genetically modified replication-defective ChAd155 vector having deleted E1/E4 gene regions and expressing the codon pair optimized rabies glycoprotein (G) (ChAd155-RGco2) (WO 2018/104919).

The degradation of ChAd155 virions in various storage media was evaluated experimentally by measuring the infectivity of the virus preparation over time at the controlled storage temperature of 4° C. Infectivity was determined using a hexon ELISA assay in HEK293 cells, which measures the expression of the viral hexon protein after infection of the cells. Stability was expressed as the ability of the virus to infect the cells. Viral infectivity was quantified as the number of infectious particles per milliliter (IP/ml) of purified, formulated virus. VP/ml of formulated virus was calculated by quantitative PCR (qPCR) using a probe hybridizing to a region in the transgene expression cassette of the viral genome.

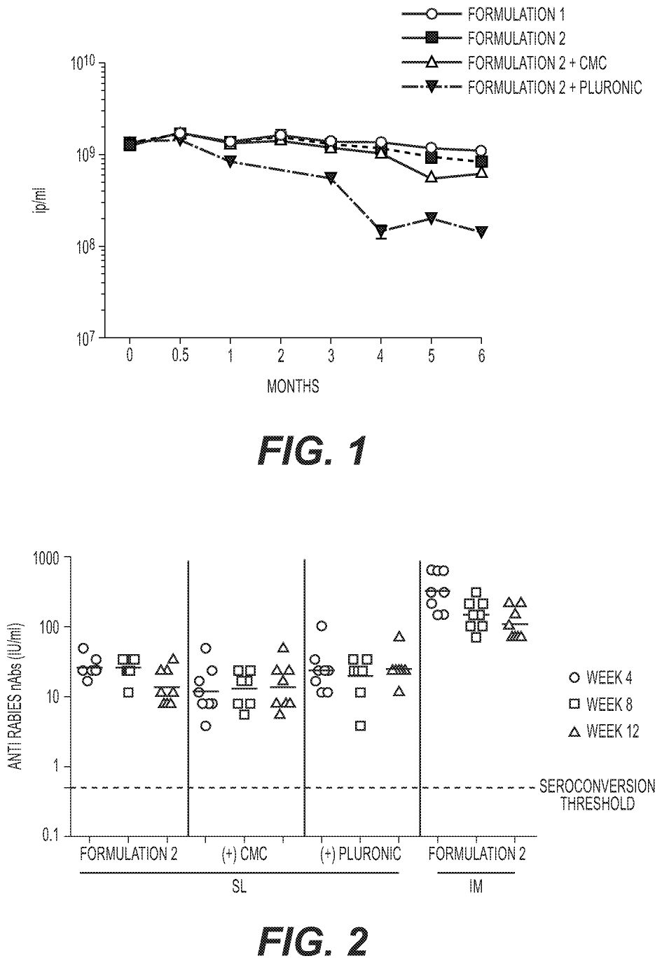

FIG. 1 shows the stability of ChAd155-RGco2 over six months at 4° C. in 10 mM Tris pH 7.4, 75 mM NaCl, 5% sucrose, 0.02% polysorbate 80, 0.1 mM EDTA, 10 mM histidine and 1 mM MgCl2 (Formulation 1) or 10 mM Tris pH 8.5, 5 mM NaCl, 10 mM histidine, 16% sucrose, 0.025% polysorbate 80, 1 mM MgCl2, 0.05 mM vitamin E succinate (VES) and 0.1% recombinant human serum albumin (rHSA) (Formulation 2) alone or with either 1.5% CMC or 20% Pluronic added. Stability was determined by measuring the number of VP and IP. As also observed for Formulation 3 (10 mM Tris pH 8.4, 5 mM NaCl, 16% trehalose, 0.02% polysorbate 80, 0.1 mM EDTA), the number of viral particles did not change over time.

FIG. 1 also demonstrates that the addition of either CMC or Poloxamer 407 did not affect viral stability. At 4° C. the virus was stable in Formulation 1 and Formulation 2 either alone or with the addition of CMC. The virus remained stable for about one month when formulated with Poloxamer 407.

The stability of the adenovirus in Formulation 2 with or without the bioadhesive reagents 1.5% CMC or 20% Poloxamer 407 after freezing at −80° C. and thawing at room temperature was measured as in the experiments above. No impact on stability was observed due to the presence of either of these bioadhesive reagents on either the number of viral particles or their infectivity.

### Example 2: In Vivo Immunogenicity of Adenovirus in Bioadhesive Formulations

To determine the impact of bioadhesives on adenoviral immunogenicity, 1×10⁹ vp ChAd155-RGco2 was formulated in Formulation 2, Formulation 2 with 1.5% CMC or Formulation 2 with 20% Poloxamer 407. Seven ul were delivered sublingually to each of six Balb/c mice. As a control, a group of mice was immunized intramuscularly with 1×10⁹ vp ChAd155-RGco2 in Formulation 2. The titers of anti-rabies viral neutralizing antibodies (VNA) in the sera was determined at four, six and eight weeks after vaccination by a fluorescent antibody virus neutralization (FAVN) test.

As shown in FIG. 2, the anti-rabies VNA titers were comparable between the three groups immunized sublingually, indicating that the presence of either CMC or Poloxamer 407 in the formulation did not negatively affect the immunological potency of the rabies vaccine. The titers of all mice immunized sublingually were well above the seroconversion threshold. As expected, intramuscular delivery induced high serum titers.

### Example 3. Effect of Known Mucosal Adjuvants on the Immunogenicity of Simian Adenovirus

Experiment 1

Sublingual administration of a simian adenovirus induced an immune response at mucosal sites and a detectable, but low systemic immune response in mice. The adjuvants LTK63 and alpha-galactosylceramide (α-GalCer) were incorporated into Formulation 2 and their effect on mucosal and systemic immune-responses determined after sublingual delivery of simian adenovirus to BALB/c mice. First, the stability of adenovirus formulated with these adjuvants was confirmed in vitro by mixing the virus with the adjuvants and incubating for two hours before infecting the cells, simulating what was done the day of immunization. Infectivity was evaluated in adherent Procell 92 cells by hexon immunostaining and it was confirmed that these adjuvants did not affect the stability of the virus.

Three groups of Balb/c mice were immunized sublingually and one group intranasally with 6.4×10⁸ vp of the adenovirus ChAd155-duaIRSV, which encodes the respiratory syncytial virus (RSV) proteins F, N and M2-1 encoded from two different expression cassettes inserted in different regions of the viral genome (PCT/EP2018/078212). Animals in group 1 received 7 ul of the virus formulated without adjuvant. Animals in group 2 received 7 ul of the virus formulated with 5 ug LTK63 and animals in group 3 received 7 ul formulated with 5 ug αGalCer. Animals in group 4 were immunized intranasally with the same dose of viral vaccine without adjuvants.

| | | | Priming | | Boosting | |
|---|---|---|---|---|---|---|
| Group | Priming Vector | Route | Formulation | Adjuvant | Vector | Route |
| 1 | 6.4 × 10⁸ vp | SL | Formulation 2 | None | 4.5 × 10⁶ pfu | SL |
| | ChAd155-dualRSV | | | | MVA-RSV | |
| 2 | 6.4 × 10⁸ vp | SL | Formulation 2 | 5 ug (7 ul) | 4.5 × 10⁶ pfu | SL |
| | ChAd155-dualRSV | | | LTK63 | MVA-RSV | |
| 3 | 6.4 × 10⁸ vp | SL | Formulation 2 | 5 ug (7 ul) | 4.5 × 10⁶ pfu | SL |
| | ChAd155-dualRSV | | | αGalCer | MVA-RSV | |
| 4 | 6.4 × 10⁸ vp | IN | Formulation 2 | None | 4.5 × 10⁶ pfu | IN |
| | ChAd155-dualRSV | | | | MVA-RSV | |

Seven weeks after the priming dose, half the animals in each group were boosted with 4.5×10⁶ pfu Modified Vaccinia Ankara virus MVA-RSV, which encodes the same RSV antigens as the simian adenoviral priming vector. The booster vector was delivered in a volume of 7 ul, without adjuvants, and the animals were sacrificed one week after boost. Saliva was collected on the day of the sacrifice by intraperitoneal injection of 10 ug pilocarpine. The mice began salivating about 20 minutes after pilocarpine administration.

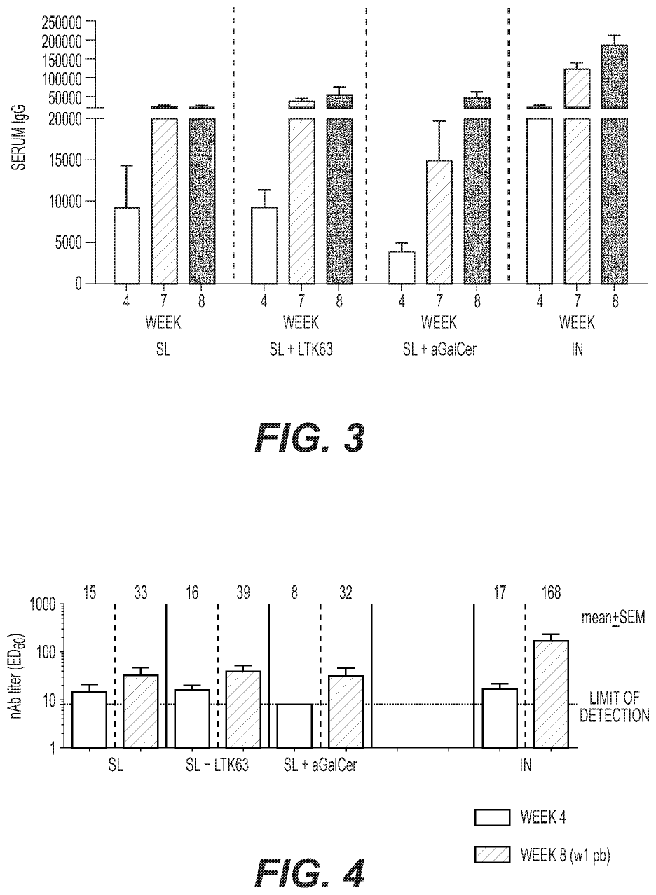

FIG. 3 demonstrates that immunization via the sublingual route induced a systemic IgG response at four weeks (post-prime), seven weeks (pre-boost) and eight weeks (post-boost). IgG was measured in the serum by ELISA on plates coated with RSV F protein and the serum titers of anti-F antibodies induced by the vaccination are expressed as endpoint titers. In animals vaccinated sublingually, boosting with MVA-RSV had little or no effect on the systemic IgG response to the unadjuvanted vector. Boosting with adjuvanted vector had a slight stimulating effect in animals vaccinated sublingually. As expected, the intranasal route was very effective at inducing a serum IgG response.

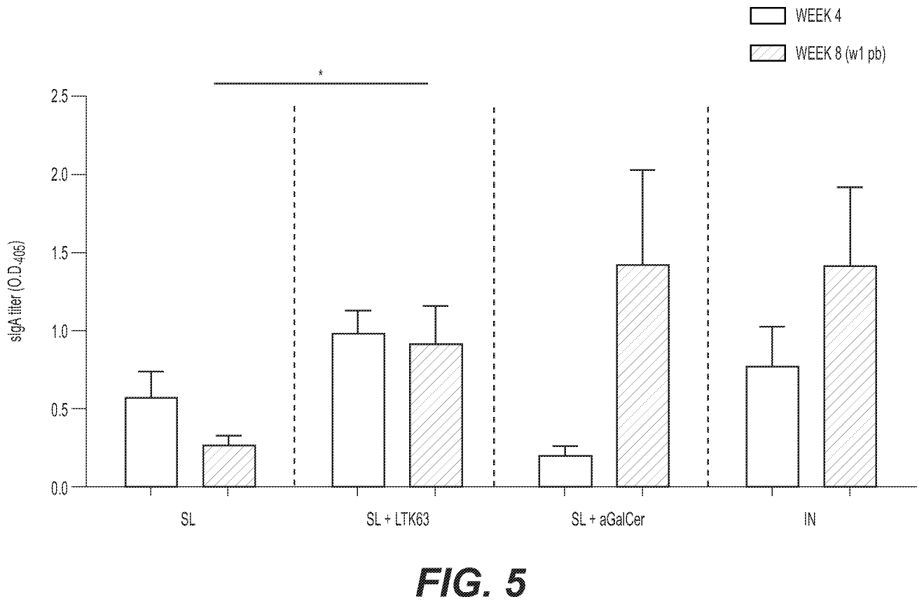

FIG. 4 shows the serum neutralizing antibody (nAb) titers post prime (week 4) and post boost (week 8). Titers were measured by an RSV-A micro-neutralization assay on Vero cells. The titer (ED₆₀) was expressed as the dilution giving 60% inhibition of plaque formation. Sublingual administration induced neutralizing, i.e., functional, antibodies to the antigen in the serum. No effect of the adjuvants was observed in the animals immunized sublingually.

FIG. 5 demonstrates that immunization via the sublingual route induced a secretory IgA (sIgA) response both at week four (post-prime) and at week eight (post-boost). Secretory IgA was measured in saliva diluted 1:6 by ELISA on plates coated with RSV F protein and expressed as optical density (O.D.₄₀₅). A sIgA response was observed in the presence and absence of adjuvant. At week four the adjuvant LTK63 increased sIgA (sIgA) in animals vaccinated sublingually to a level comparable to that of animals vaccinated intranasally. After boosting, the level of sIgA remained constant. At week four, the adjuvant α-GalCer did not increase sIgA in animals vaccinated sublingually, however, after boosting, a robust sIgA response was observed comparable to that of animals vaccinated intranasally. Both LTK63 and α-GalCer had an adjuventing effect but the effect was not strong enough to overcome the individual variation between the mice.

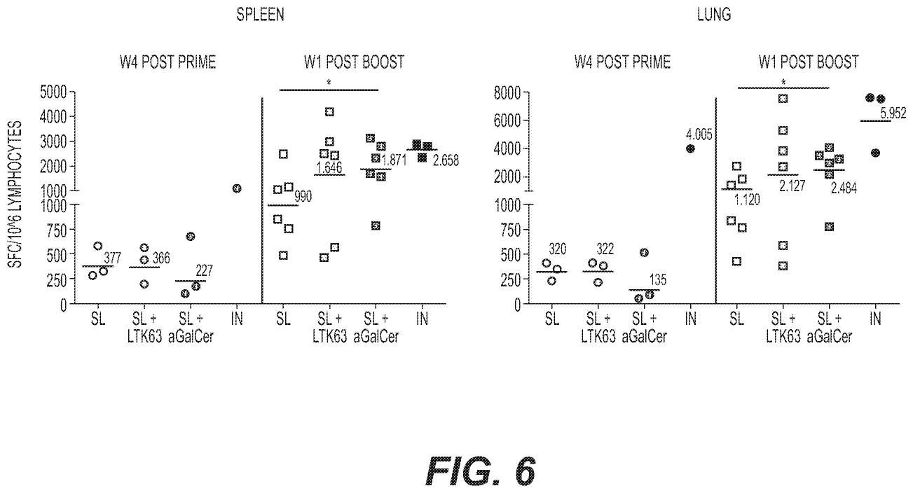

Sublingual administration of simian adenovirus stimulates an antigen specific T cell response, which is amplified both by boosting and by the adjuvants LTK63 and α-GalCer. FIG. 6 shows the systemic (spleen) and local (lung) RSV specific T cell responses induced by the vaccination, measured using an IFNγ ELISpot assay on splenocytes and lung homogenates at four weeks post prime and one-week post boost. IFNγ ELISpot analysis enumerates the antigen specific T cells that secrete the cytokine IFNγ using a capture antibody to IFN-γ bound to a membrane sandwiched with a complex of a biotinylated Ab and streptavidin conjugated to alkaline phosphatase, resulting in the precipitation of a chromogenic substrate that generates a spot on the membrane where the antigen specific cell was located.

As shown in FIG. 6, sublingual administration of adenovirus induced an antigen specific T cell response in both the spleen and lung at four weeks (post-prime). Formulating the adenovirus with either LTK63 or α-GalCer resulted in a much greater expansion of vaccine specific T cells after boosting, both systemically (spleen) and locally (lungs).

Experiment 2

A similar experiment was then performed with the addition of the adjuvant interleukin 1 beta (IL1β) incorporated into a transgene. Four groups of CB6 mice were immunized sublingually, one group intranasally and one group intramuscularly with 1.0×10⁹ vp of the adenovirus ChAd155-duaIRSV or ChAd155-duaIRSV with IL1β inserted into a transgene cassette (ChAd155-dual RSV-IL1β), as shown in the table below. All animals were boosted at week 12 with 4.5×10⁶ pfu MVA-RSV.

| | | | Priming | | Boosting | |
|---|---|---|---|---|---|---|
| Group | Priming Vector | Route | Formulation | Adjuvant | Vector | Route |
| 1 | 1.0 × 10⁹ vp | SL | Formulation 2 | None | 4.5 × 10⁶ pfu | SL |
| | ChAd155-dualRSV | | | | MVA-dual | |
| | | | | | RSV | |
| 2 | 1.0 × 10⁹ vp | SL | Formulation 2 | 10 ug | 4.5 × 10⁶ pfu | SL |
| | ChAd155-dualRSV | | | LTK63 | MVA-dual | |
| | | | | | RSV | |
| 3 | 1.0 × 10⁹ vp | SL | Formulation 2 | 4 ug | 4.5 × 10⁶ pfu | SL |
| | ChAd155-dualRSV | | | αGalCer | MVA-dual | |
| | | | | | RSV | |
| 4 | 1.0 × 10⁸ vp | SL | Formulation 2 | Transgenic | 4.5 × 10⁶ pfu | SL |
| | ChAd155-dualRSV- | | | IL1β | MVA-dual | |
| | IL1b | | | | RSV | |
| 5 | 1.0 × 10⁹ vp | IN | Formulation 2 | None | 4.5 × 10⁶ pfu | IN |
| | ChAd155-dualRSV | | | | MVA-RSV | |
| 6 | 1.0 × 10⁹ vp | IM | Formulation 2 | None | 4.5 × 10⁶ pfu | IM |
| | ChAd155-dualRSV | | | | MVA-RSV | |

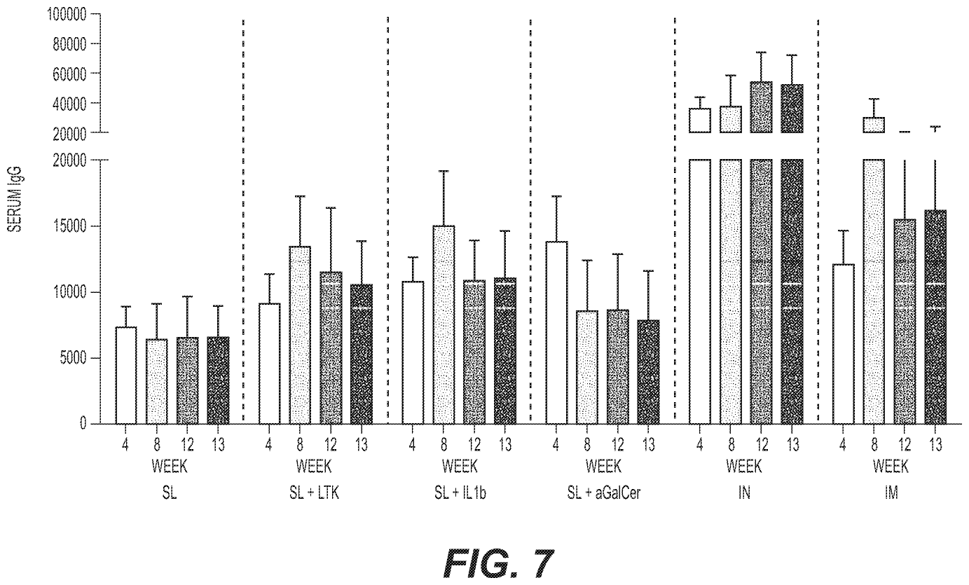

FIG. 7 demonstrates that immunization via the sublingual route induced a detectable systemic IgG response. Serum IgG was measured at weeks four, eight, twelve (pre-boost) and thirteen (post-boost) by an IgG ELISA on plates coated with RSV F protein. As in Experiment 1, no clear effect of the adjuvants was observed and boosting with MVA-RSV had little or no effect on the systemic IgG response. As expected, the intranasal and intramuscular routes were very effective at inducing serum antibody responses.

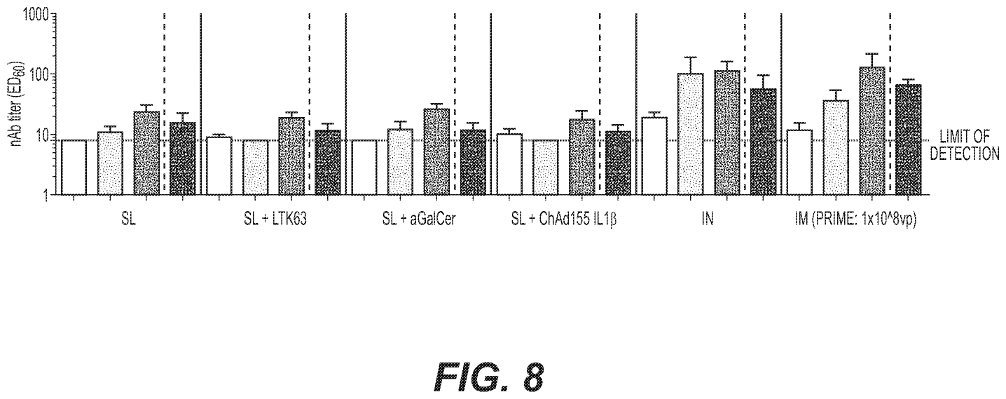

FIG. 8 demonstrates that sublingual administration induced neutralizing, i.e., functional, antibodies to the antigen in the serum. Neutralizing antibodies were measured and expressed as in Experiment 1. No effect of the adjuvants on systemic neutralizing antibodies was observed in the animals immunized sublingually.

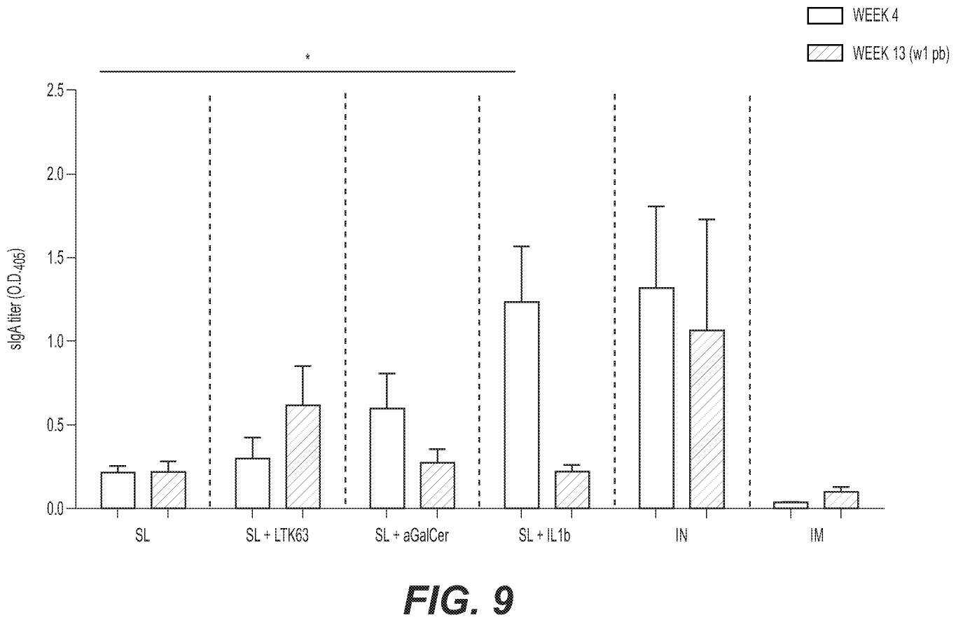

FIG. 9 demonstrates that immunization via the sublingual route induced a secretory IgA response both at week four (post-prime) and at week thirteen (post-boost). Secretory IgA in saliva was measured and expressed as in Experiment 1. The adjuvants α-GalCer and IL1β increased sIgA production post-prime. Sublingual administration of adenovirus adjuvented with IL1β resulted in secretory IgA salivary levels equal to those induced by unadjuvanted adenovirus administered intranasally. LTK63 increased sIgA production post boost. Boosting with MVA did not increase sIgA production in the absence of adjuvant or in the presence of α-GalCer or IL1β.As expected, intramuscular administration did not result in salivary IgA production.

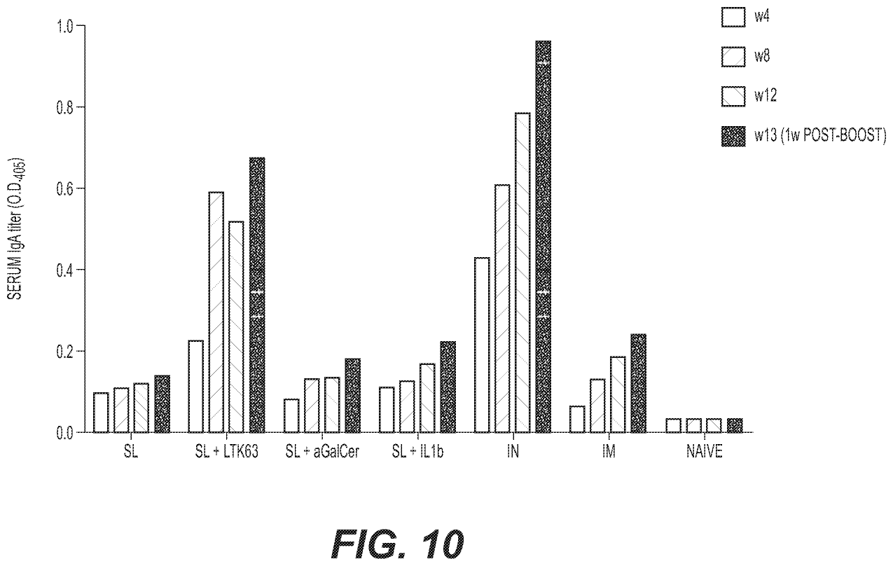

FIG. 10 demonstrates that LTK63 increases serum IgA production to levels comparable to intranasal immunization. Serum IgA diluted 1:45 was measured by F-protein ELISA after depleting the interfering serum IgG by treatment with protein G agarose. The sera were incubated at room temperature for two hours with the resin, and after centrifugation the supernatant was analysed for specific IgA content. Following sublingual administration, LTK63 increased systemic IgA production to levels comparable to intranasal administration at weeks 4, 8 and 12 pre-boosts and at week 13, one week post-boost.

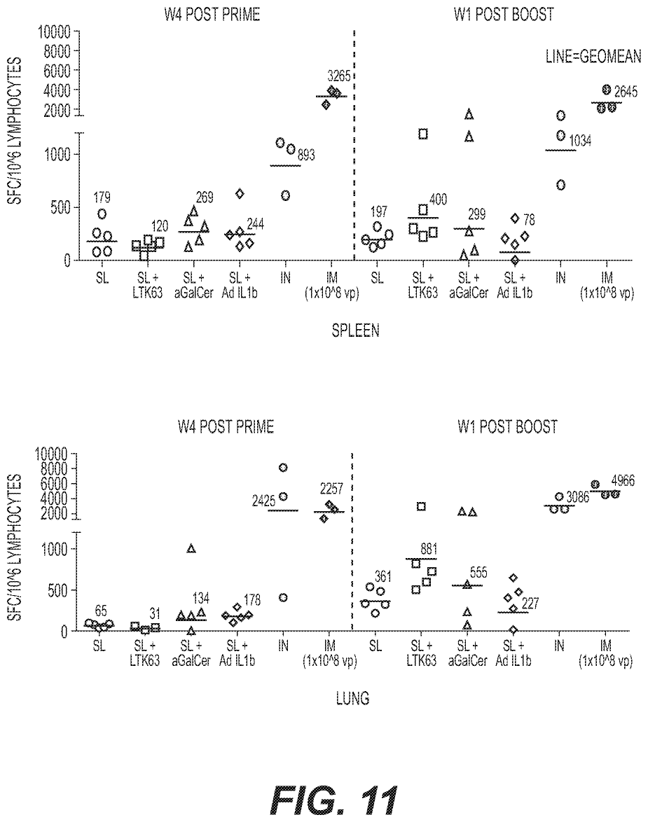

FIG. 11 shows the systemic and local RSV specific T cell responses induced by the vaccination, measured using an IFNγ ELISpot assay on splenocytes and lung homogenates at four weeks (post prime) and one-week post boost. As shown in FIG. 11, the formulation of a simian adenovirus with adjuvants upon priming led to a greater expansion of vaccine specific T cells in the lung after boosting. The expansion of the T cell response elicited by the adjuvants was especially evident locally, i.e., in the lung,

In conclusion, a simian adenovirus vaccine encoding an immunogenic transgene and a bioadhesive excipient in an aqueous formulation delivered by the mucosal route can induce secretory IgA, a systemic antibody response and vaccine specific T cell response both systemically and locally.

1. A composition comprising a recombinant simian adenovirus encoding an immunogenic transgene and a bioadhesive excipient in an aqueous formulation.

2. The composition of claim 1, wherein the bioadhesive is selected from polyoxyethylene, poly(ethylene glycol) (PEG); poly(vinyl pyrrolidone) (PVP); poly(hydroxyethyl methacrylate) (PHEMA); a pluronic; a polyacrylate; a carbomer; polycarbophil; hyaluronic acid; a chitosan; an alginate; guar gum; carrageenan; and a polymer derived from cellulose.

3. The composition of claim 1 wherein the bioadhesive is a pluronic and is selected from Pluronic F-68, Pluronic 127 and Poloxamer 407.

4. The composition of claim 1, wherein the pluronic is Poloxamer 407.

5. The composition of claim 1, wherein the bioadhesive is derived from cellulose and is selected from carboxymethylcellulose (CMC), microcrystalline cellulose, oxidized regenerated cellulose, hydroxyethyl cellulose (HEC), hydroxypropyl cellulose (HPC), methylcellulose and sodium carboxymethylcellulose.

6. The composition of claim 1, wherein the bioadhesive derived from cellulose is carboxymethylcellulose (CMC).

7. The composition of claim 1, wherein the composition further comprises Tris, NaCl, an amorphous sugar and a surfactant.

8. The composition of claim 1, wherein the composition further comprises one or more of a bivalent metal ion, EDTA, histidine, ethanol, Vitamin E succinate and albumin.

9. The composition of claim 1, further comprising Tris, NaCl, an amorphous sugar and a polysorbate surfactant.

10. The composition of claim 1, further comprising LTK63 or alpha-galactosylceramide (α-GalCer).

11. The composition of claim 1, wherein the immunogenic transgene comprises an interleukin 1 beta (IL1β) gene.

12-22. (canceled)

23. A method of inducing an immune response in a mammal, which comprises by administering a recombinant simian adenovirus encoding an immunogenic transgene and a bioadhesive excipient in an aqueous formulation to the mucosa of the mammal.

24. (canceled)

25. The method of claim 23, wherein the formulation is delivered to the buccal, colorectal, under-eyelid, gastrointestinal, lung, nasal, ocular, sublingual or vaginal mucosa.

26. The method of claim 23, wherein the bioadhesive is selected from polyoxyethylene, poly(ethylene glycol) (PEG); poly(vinyl pyrrolidone) (PVP); poly(hydroxyethyl methacrylate) (PHEMA); a pluronic; a polyacrylate; a carbomer; polycarbophil; hyaluronic acid; a chitosan; an alginate; guar gum; carrageenan; and a polymer derived from cellulose.

27. The method of claim 23, wherein the composition further comprises one or more of NaCl, an amorphous sugar, a surfactant, a bivalent metal ion, EDTA, histidine, ethanol, Vitamin E succinate and albumin.

28. The method of claim 23, wherein the bioadhesive is a pluronic.

29. The method of claim 28, wherein the pluronic is Poloxamer 407.

30. The method of claim 23, wherein the bioadhesive is a polymer derived from cellulose.

31. The method of claim 30, wherein the polymer derived from cellulose is carboxymethylcellulose (CMC).

32. The method or use of claim 23, wherein the composition further comprises LTK63 or alpha-galactosylceramide (α-GalCer).

33. The method of claim 23, wherein the immunogenic transgene comprises an interleukin 1 beta (IL1β) gene.
